(19) United States
(12) Patent Application Publication
(10) Pub. No.: US 2003/0124722 A1
(43) Pub. Date: Jul. 3, 2003

(54) Chamber for artificial organ

(76) Inventors: Hisako Ohgawara, Tokyo (JP); Kosiro Sato, Tokyo (JP)

Correspondence Address: CARELLA, BYRNE, BAIN, GILFILLAN,, CECCHI, STEWART & OLSTEIN, 6 Becker Farm Road, Roseland, NJ, 07068, US

(21) Appl. No.: 10/324,395

(22) Filed: Dec. 20, 2002

(30) Foreign Application Priority Data
Dec. 28, 2001 (JP) JP 2001-400511

(51) Int. Cl. C12N 5/08; C12M 1/00

(52) U.S. Cl. 435/366; 435/293.1

(57) ABSTRACT

A chamber is arranged as follows to prevent deteriorating functions of pancreatic cells, namely, reducing secretory amount of insulin. The chamber for artificial organs comprises, a silicone rubber ring and two porous polymer membranes adhered to both side of the silicone ring so as to form a vessel. Pancreatic cells having functions capable of substituting for a pancreas which loses its functions and a cell culture scaffold are filled in the vessel. Spongy chitin is also filled in the vessel as a carrier of the pancreatic cells.

### BACKGROUND OF THE INVENTION

1. Field of the Invention

The present invention relates to a chamber for artificial organs used in place of a secretory organ of which functions are totally or partially lost owing to diseases or genetic reasons

2. Brief description of the Related Art

As conventional examples such chambers for artificial organs, Japanese utility model registered No.6-13739 and Japanese patent laid open No.10-57405 disclose a container made from a silicone rubber ring adhered two polymer porous membranes (immuno-isolative membranes) to its both ends, in which insulin secretory cells and a culture scaffold (substrate) for the cells are filled.

When this chamber is applied to test animals such as rats or small dogs, no foreign body rejecting reactions are observed even it has been kept in the bodies of such test animals. Moreover, since blood vessels are newly formed around chamber, insulin can be efficiently circulated in the bodies, as a result sugar content in blood is successfully reduced.

As the inventors reported in a Japanese medical journal “Igaku no Ayumi” (vol.196 No.5, February, 2001), since insulin secretory cells are merely dispersed on the culture scaffold (substrate) in the conventional chamber, the base swells and re-coagulates as the chamber has been used for a long period. Which not only causes gradual deterioration in functions (secreting volume of insulin) of the cells, but also breaks membranes.

If a larger sized chamber is applied to a test animal in accordance with its body weight, the problems mentioned above are solved. Although silicone rubber and membranes, used as components for the chamber, have good adaptability to living bodies, the chamber is still a foreign body. As a result, the larger the chamber is, the stronger the rejecting reaction becomes, in other words, the higher the risk against the living body increases.

Insulin cell separating methods are disclosed, for example, in Japanese laid open patent Nos. 7-99970 and 2001-231548. However, since collecting yields are so low in these disclosed methods that it is difficult to accumulate sufficient numbers of cells from one body, cells used in one chamber have to be collected from a plurality of bodies and be mixed. It is inevitable that activity of the chamber containing cells from the plurality of bodies is lower than that of a chamber containing cells from only one body.

### SUMMARY OF THE INVENTION

The present invention is carried out in order to solve the above-mentioned problems and to provide a chamber for artificial organs capable of preventing functions of cells from deteriorating, and also to provide a chamber for artificial organs containing cells collected from one living animal body.

The present invention provides chambers for artificial organs described in (1) to (3).

(1) A chamber for artificial organs comprises a silicone rubber ring and two porous polymer membranes adhered to both side of the silicone ring so as to form a vessel. Cells having functions capable of substituting for a secretory organ which loses its functions and a cell culture scaffold are filled in the vessel. And chitin is filled in the vessel as a carrier of the cells. (Hereinafter referred as a first chamber.)

(2) The chamber for artificial organs according to (1), the amount of the chitin is determined so as to stabilize the shape of the vessel due to swelling of chitin by water containing the cell culture scaffold. (Hereinafter referred as a second chamber.)

(3) The chamber for artificial organs according to (1), the chitin formed in a sheet, spongy or fibrous shape is employed.

Cells to be filled in the above-mentioned vessel are cells separated from secretory organs such as pancreas, kidney, adrenal cortex or suprarenal medulla, thymus, thyroid, hypophysis, pineal body and the like.

When cells separated animal bodies are filled in the chambers by the present invention, it is desirable to use cells separated from one animal body so as to enhance activities of the chambers and to control quality of the chambers.

Any cell separating method capable of yielding at least more than 1.5×10⁷, preferably more than 1.7×10⁷ and more preferably more than 2.0×10⁷ cells is acceptable as a separating method for the present invention. A separating method as follows is one of the examples.

A pancreas originated from one animal body is swelled by a circulating physiologically acceptable and enzyme free solution. The swelled pancreas is cut into fine pieces. Pancreatic secretory cells are separated from the finely cut pancreas by any one of the various separating apparatuses. The physiologically acceptable and enzyme free solution can be introduced to any part of the pancreas. However, it is preferable to introduce the solution via an opening at the duodenum side through the pancreatic duct, because the whole pancreas is swelled and can be utilized effectively by this circulating method. As a result much more cells can be collected from the pancreas originated from one body.

The chambers for artificial organs provided in the above-mentioned ways, have the same functions and activities as a pancreas in a living body. Therefore the chambers by the present invention can be effectively used pancreatic drug tests and toxicity tests. The chambers can be used as artificial organs for treating diabetes, pancreas cancer and other pancreatic diseases.

### BRIEF DESCRIPTION OF THE DRAWINGS

FIG. 1 is a perspective view of a chamber for artificial organs by the present embodiment.

FIG. 2 is an enlarged cross-sectional view along A-A plane.

### DETAILED DESCRIPTION OF THE PREFERRED EMBODIMENTS

Hereinafter embodiments by the present invention are explained.

(Embodiment 1)

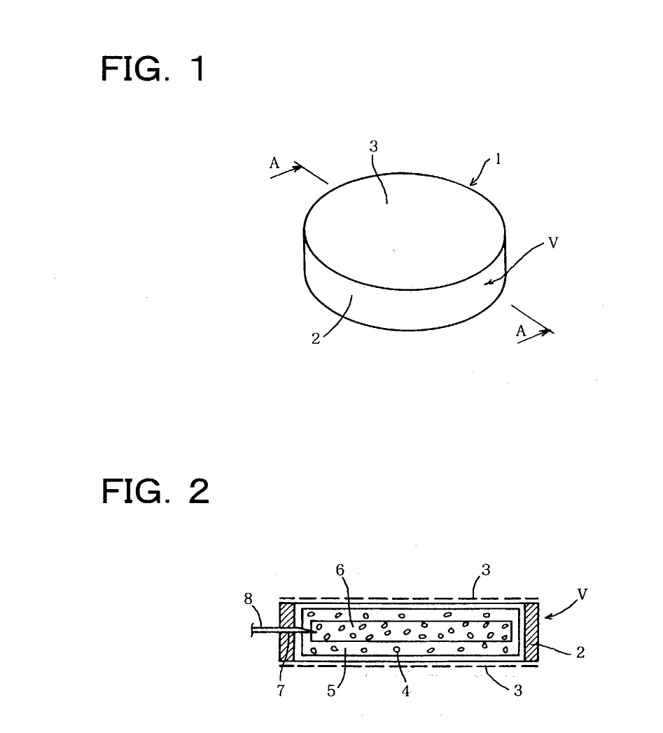

FIG. 1 is the perspective view of a chamber for artificial organs by the present embodiment and FIG. 2 is the enlarged sectional view along A-A plane.

In these figures, a reference numeral “1” is a chamber for artificial organs. The chamber consists of; a silicone rubber ring 2 with an outer diameter of ca. 50 mm, an inner diameter of ca. 30 mm and a thickness of ca. 2 mm and two immuno-isolative membranes 3 with 6 μm thickness on average made from polycarbonate resin adhered to both sides of the silicone rubber ring 2. A vessel V is formed by the ring 2 and membranes 3. Pancreas cells 4 isolated from a porcine pancreas, a cell culture scaffold (substrate) 5 and a disk shaped spongy chitin 6 are enclosed in the vessel V.

Procedures to enclose the pancreas cells 4, the cell culture scaffold (substrate) 5 and the disk shaped spongy chitin 6 are carried out as follows.

(1) During forming the vessel V, the disk shaped spongy chitin 6 with a diameter of ca. 30 mm and a thickness of ca. 2 mm is placed in the container V.

(2) The pancreatic cells 4 together with the substrate 5 are injected into the vessel V little by little via an injection needle 8 inserted in a hole 7 formed on the wall of the ring 2 so as to impregnate with the chitin 6. An apparent volume change in the spongy chitin 6 is not observed. However, when a disk shaped sheet chitin with a diameter of ca. 30 mm and a thickness of ca. 0.6 mm is employed, the disk shaped sheet chitin swells ca. three times.

(3) After a predetermined volume is injected, the vessel V is left in a culture solution for a while so as to stabilize the disk shaped chitin.

Chambers for artificial organs formed in the above-mentioned way are implanted in abdominal cavities of two apes.

One of the apes has been suffering from lipoatrophic diabetes for a long period and been showing a symptom of grave weight reduction. Before implanting the chamber, an insulin value in blood of the ape is 3.4 μU/ml and a sugar value in the blood shows a high value of 300 mg/dl, which show similar values to those of type I diabetes.

Although an insulin value of the ape 1 week after the implantation was raised up to 36.3 μU/ml, still a symptom of lipoatrophic diabetes was observed, and a sugar value in blood was 200 mg/dl and a strong insulin resistance was observed. One month after the implantation the insulin resistance is reduced and a sugar value at a hunger state is 138 mg/dl, which indicates that insulin from implanted cells secretes well. Two month after the implantation a sugar value is constantly reduced to less than 100 mg/dl so that effects of the chamber for artificial organs by the present embodiment are proved.

Another implanted ape has been suffering from diabetes for a short period, but secreted insulin volume has been lowered due to obesity (a temporal high insulin resistance due to high insulin secretion) and a high sugar value is observed, which indicates type II diabetes.

The sugar value of another ape before the implantation is 300 mg/dl, but it is reduced to 118-165 mg/dl one month after the implantation, thus the effectiveness of the chamber for artificial organs are also proved.

(Embodiment 2)

20 G angiography catheter is inserted into an extracted matured porcine pancreas via an opening at the duodenum side through the accessory pancreatic duct. 3% new born bovine serum (produced by GIBCO), 5.5 mM (“M” means mol per liter) D-glucose (produced by Wako) and 0.01M phosphate buffer solution (produced by Sigma Co., hereinafter referred as “PBS”) including 10 mM nicotine amide (produced by Wako) cooled at 4° C., are poured into the porcine pancreas via the angiography catheter. The swelled porcine pancreas is washed with super acid solution and is stored in a cooled RPMI-1640 solution (produced by GIBCO) including 10 mM nicotine amide.

After removing ambient tissues, and pancreatic ducts, fat and the like inside the stored porcine pancreas, the stored porcine pancreas is cut into square pieces with 2 cm by 2 cm. The square pieces are cut into fine pieces by a mechanical chopper (produced by Microtec, TS-001). Pancreas cells are extracted by using a rotary cell extraction apparatus. The apparatus consists of a rotating stainless mesh for stirring pancreatic tissues and a water tank which surrounds the rotating stainless mesh. After filling the finely cut porcine pancreas pieces in the stainless mesh and filling the PBS kept at a room temperature in the water tank, the apparatus is stirred. After stirring for a predetermined period, dispersed porcine pancreas cells in the PBS by an autodigestion, are collected. The PBS is filled to the tank and the apparatus is stirred again. In this way the tank is stirred 7 times for 5, 10, 15, 15, 15, 15 and 15 minutes respectively.

A solution including collected cells is centrifuged at 3000 rpm for 1 minute at 2° C. After centrifuging sediments are suspended in cooled PBS, the suspended solution is centrifuged at 1200 rpm for 2 minutes at 2° C. Further after centrifuging, sediments are suspended in cooled PBS and the suspended solution is centrifuged at 1800 rpm for 1 minutes at 2° C.

Sediments obtained by the last centrifuging procedure are suspended in PBS. After stratifying the suspended cooled PBS solution of 15 ml over Histopaque-1077 solution of 10 ml, the stratified solution is centrifuged at 1800 rpm for 10 minutes. Pancreas secreting cells form a vague white thin layer at a boundary between the suspended PBS solution and Histopaque-1077. The formed layer by the cells is collected by a pipet. The collected cells are put into RPMI-1640 culture solution (produced by GIBCO) including 10% heat inactivated fetal bovine serum (hereinafter referred as “FBS”), 11 mM D-glucose and 10 mM nicotine amide and are washed two times by centrifuging 1200 rpm for 3 minutes.

a) Yield of Porcine Secreting Cell

Cells obtained by the above-mentioned centrifuging procedures are counted and at the same time after staining cells by dithizone solution (DTZ), a DTZ positive cell (βcells) rate and a DTZ positive cell number are calculated.

b) Adhered and Grew Cell Number

Cells obtained by the centrifuging procedures are cultured by RPMI-1640 culture solution including 10% FBS, 11 mM D-glucose and 10 mM nicotine amide in a cell culturing flask (produced by Sumitomo Bakelite, MS-22050) for 7 days. After separating porcine secretory cells adhered to and grew in the flask by a mixed solution of 0.005% EDTA (produced by ICN) and 0.125% trypsin (produced by GIBCO), the number of separated cells are counted. Part of separated cells are used for histologic observations.

c) Evaluation of Functions of Pancreatic Secretory Cell

Viability of the pancreatic secretory cells are evaluated by measuring insulin secretory activities against glucose stimulation. Immediately after separating, the separated pancreatic secretory cells are divided into 6 groups so as to contain 2×10⁵ cells in each group and put into 6 test cells. After culturing 6 samples for 7 days in a static state, evaluation of activities of respective samples are carried out by utilizing insulin loading tests (static incubation rest) in the following manner.

As pre-incubations samples are cultured in RPMI-1640 culture solution including 3.3 mM D-glucose for 60 minutes and cultured in RPMI-1640 culture solution including 3.3 mM D-glucose and 0.1% bovine serum albumin (BSA, produced by Wako) for 60 minutes. Then culture a low glucose culture solution consisting of RPMI-1640 including 3.3 mM D-glucose and 0.1% BSA and a high glucose culture solution consisting of RPMI-1640 including 10 mM D-glucose and 0.1% BSA are prepared. Insulin concentrations in respective samples are measured and determined secreted amounts of insulin by using ELISA after culturing the samples consequently in order of low glucose culture solution, high glucose culture solution and low glucose culture solution for 60 minutes respectively.

Obtained results are as follows.

1) Yield of porcine secretory cell: 2.97±0.59×10⁷

2) DMZ positive cell rate: 55.9±12.1%

3) DMZ positive cell number: 1.64±0.36×10⁷

4) Adhered and grew cell number: 1.07±0.26×10⁷

### REFERENCE EXAMPLE

An extracted pancreas is treated by a conventional method. Namely, the pancreas is stored in cooled RPMI-1640 culture solution including 10 mM nicotine amide and immediately washed by super acid solution. After removing connective tissues, blood vessels lymph nodes fats and the like around the pancreas, a cooled phosphate buffer solution including 3% new born bovine serum is injected into pancreatic tissues by using 19 G syringe needle (multiple injection method). Other procedures are same as the Embodiment 2. 6 samples are prepared and evaluated.

The results of the reference example are as follows.

1) Yield of porcine secretory cell: 0.89±0.15×107

This result indicates that the yield by the Embodiment 2 is ca. 3 times higher the conventional method.

2) DMZ positive cell rate: 41.5±6.2%

3) DMZ positive cell number: 0.36±0.09×10⁷

4) Adhered and grew cell number: 0.36±0.03×10⁷

At the static incubation test stage, since the numbers of cell in test cells are the same each other, no significant differences are observed between Embodiment 2 and the reference example.

The following effects are attained by the present invention.

(1) Since chitin is filled with culture solution in the first, second and third chambers, cell culture scaffolds are prevented from swelling and re-coagulation so that damages of cells and deterioration of cell functions are prevented.

(2) In the second chamber, since an amount of chitin to stabilize the shape of the vessel due to swelled chitin by water containing the cell culture scaffold, the vessel can be kept in a stable state without deformation.

(3) In the third chamber, since sheet formed chitin, spongy chitin or fibrous chitin is used, it is easy to handle the chitin when fabricating the chamber for artificial organs.

(4) Since cells separated by procedures described in embodiment 2 indicate excellent yield and activities in secretory cells, the inventors believe embodiment 2 are useful procedures.

What is claimed is:

1. A chamber for artificial organs comprising:
a silicone rubber ring; and
two porous polymer membranes adhered to both side of said silicone ring so as to form a vessel, wherein:
cells having functions capable of substituting for a secretory organ which loses its functions, and a cell culture scaffold are filled in said vessel, wherein:
chitin is filled in said vessel as a carrier of said cells.

2. The chamber for artificial organs according to claim 1, wherein the amount of said filled chitin is determined so as to stabilize the shape of said vessel due to said swelled chitin by water containing said cell culture scaffold.

3. The chamber for artificial organs according to claim 1, wherein said chitin formed in a sheet, spongy or fibrous shape is used.

4. The chamber for artificial organs according to either one of claims 1 to 3, wherein said filled cells capable of substituting for a secretory organ are originated from the same animal body.

5. The chamber for artificial organs according to either one of claims 1 to 4, wherein said filled cells capable of substituting for a secretory organ are porcine pancreatic secretory cells.

6. A method for separating pancreatic secretory cells from a pancreas originated one animal body comprising steps of:
circulating a physiologically acceptable and enzyme free solution in said pancreas so as to swell said pancreas;
cutting said swelled pancreas into fine pieces, and
separating pancreatic secretory cells from said finely cut pancreas, wherein:
the number of separate cells from one porcine pancreatic secretory cells is more than 1.5×10⁷.

7. A drug effect and toxicity evaluating system utilizing either one of the chambers for artificial organs according to claims 1 to 5.
